(19) United States
(12) Patent Application Publication
(10) Pub. No.: US 2009/0048586 A1
(43) Pub. Date: Feb. 19, 2009

(54) PRECISE DISRUPTION OF TISSUE IN RETINAL AND PRERETINAL STRUCTURES

(75) Inventors: Ronald R. Krueger, Cleveland, OH (US); Holger Lubatschowski, Gehrden (DE)

Correspondence Address: TAROLLI, SUNDHEIM, COVELL & TUMMINO L.L.P., 1300 EAST NINTH STREET, SUITE 1700, CLEVEVLAND, OH, 44114, US

(73) Assignee: The Cleveland Clinic Foundation

(21) Appl. No.: 12/191,663

(22) Filed: Aug. 14, 2008

Related U.S. Application Data
(60) Provisional application No. 60/955,976, filed on Aug. 15, 2007.

(51) Int. Cl. A61F 9/008; A61B 18/20

(52) U.S. Cl. 606/5

(57) ABSTRACT

Systems and methods are provided for disrupting tissue within preretinal or retinal structures of an eye. At least one femtosecond laser pulse is directed through the cornea of the eye to a target location. The at least one femtosecond laser pulse has sufficient intensity to induce nonlinear absorption in tissue within the target location. The at least one laser pulse is corrected at an adaptive optical element as to substantially reduce dispersion and aberration of the at least one laser pulse due to changes in the wavefront profile associated with the laser pulse due to travel through eye tissue between the surface of the eye and the target location. At least the target location is imaged to produce an in vivo image of the target location. The adaptive optical element is adjusted according to distortion detected in a reflected wavefront.

### RELATED APPLICATION

This application claims priority to the filing date of U.S. Provisional Application No. 60/955,976, filed Aug. 15, 2007, the subject matter of which is incorporated herein by reference.

### FIELD OF THE INVENTION

The present invention relates to a system and method for performing medical procedures in the retinal and preretinal regions of an eye, in particular, is directed to systems and methods for cutting tissue around the retina of the eye by photodisruption.

### BACKGROUND OF THE INVENTION

FIG. 1 illustrates a sketch (10) of a vitreoretinal traction, specifically a posterior hyaloid traction exhibiting retinoschisis. In this condition, a portion of the vitreous tissue (12) of the eye has adhered to the retinal tissue (14), causing the retinal tissue to lift away from the underlying retinal pigment epithelium (16). Left untreated, vitreoretinal traction can lead to damage to the retina as well as retinal detachment.

The traditional method for treating vitreoretinal traction is a posterior vitrectomy, an invasive procedure in which the band of vitreous tissue (18) in traction with the retina is removed. The rate of post-operative morbidity in this procedure is significant, with a high incidence of cataract formation due to the invasive nature of the procedure. As a result, even when a vitreoretinal traction condition is identified, it is common to simply monitor the procedure until a significant degradation of a patient's eyesight is observed. Until this point, the risk of damage to the retina and cataract formation is too high to justify the procedure.

### SUMMARY OF THE INVENTION

In accordance with an aspect of the present invention, a method is provided for disrupting tissue within preretinal and retinal structures of an eye. At least one femtosecond laser pulse is directed through the cornea of the eye to a target location. The at least one femtosecond laser pulse has sufficient intensity to induce nonlinear absorption in tissue within the target location. The at least one laser pulse is corrected at an adaptive optical element as to substantially reduce dispersion and aberrations of the at least one laser pulse due to changes of the wavefront of the laser pulse while it is transmitted within eye tissue between the surface of the eye and the target location. The optical element consists of a deformable mirror and/or a phase plate. At least the target location is imaged to produce an in vivo image of the target location. The adaptive optical element is adjusted according to distortion detected in a reflected wavefront.

In accordance with an aspect of the present invention, a system is provided for precisely disrupting tissue within a preretinal or retinal structure of the eye. A femtosecond laser is configured to direct laser pulses having a duration on the order of femtoseconds through the cornea of the eye to a target, location in the preretinal vitreous tissue or retinal microstructures. An imaging element is operative to image at least the target location to produce an in vivo image of the target location. An adaptive optical element is operative to correct laser pulses from the laser apparatus as to substantially compensate for the effects of optical aberrations and dispersion within eye tissue anterior of the target location.

In accordance with yet another aspect of the present invention, an apparatus is provided for precisely disrupting tissue within a preretinal or retinal structure of the eye. A femtosecond laser configured to direct laser pulses having a duration on the order of femtoseconds through the cornea of the eye to a target location in the preretinal vitreous tissue or retinal structures. An adaptive optical element is operative to correct laser pulses from the laser apparatus as to substantially mitigate the effects of optical aberrations within eye tissue anterior of the target location. The adaptive optical element can include an adaptive element that can be manipulated as to adjust its optical properties, such that one or more properties of the laser pulse will be altered through interaction with the adaptive element. A wavefront sensor detects distortion in wavefronts reflected from the eye to provide an indication of optical aberrations within the eye, such that the optical properties of the adaptive element are altered in accordance, with the output of the wavefront sensor.

### BRIEF DESCRIPTION OF THE DRAWINGS

The foregoing and other features of the present invention will become apparent to those skilled in the art to which the present invention relates upon reading the following description with reference to the accompanying drawings, in which:

FIG. 1 illustrates a sketch of a vitreoretinal traction;

FIG. 2 illustrates a system for precisely applying transcorneal laser pulses to preretinal or retinal tissue at a target location in accordance with an aspect of the present invention;

FIG. 3 illustrates the visual acuity of an arbitrary eye as a function of the pupil size.

FIG. 4 illustrates one example of an implementation of a system for noninvasive ablation of preretinal or retinal tissue within an eye via a transcorneal laser pulse; and

FIG. 5 illustrates a method for precisely disrupting tissue within the preretinal and retinal regions of the eye in accordance with an aspect of the present invention.

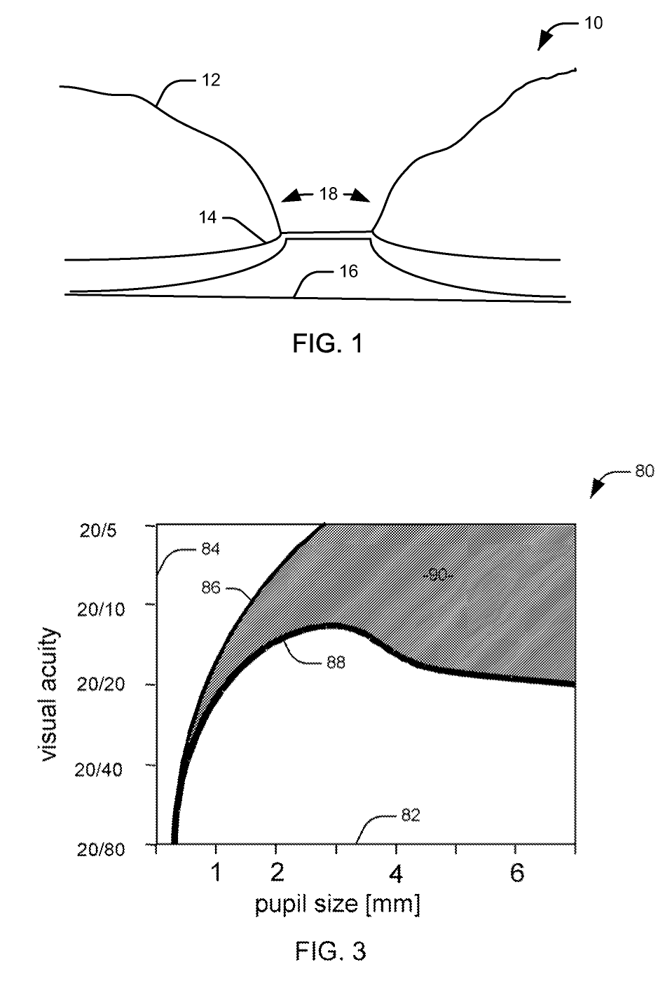

### DETAILED DESCRIPTION OF THE INVENTION

In accordance with an aspect of the present invention, systems and methods are provided for precisely disrupting preretinal and retinal tissue via transcorneal, ultra-short duration laser pulses. Specifically, the claimed systems and method precisely focus transcorneal laser pulses directed at the posterior portion of the eye to minimize damage to the retina from a given pulse, limiting post-surgical morbidty and the incidence of cataracts. This can be accomplished by using a high intensity, short duration laser pulse, on the order of femtoseconds, preferably between 10 and 1000 femtoseconds, and adaptively correcting the pulse for defects within the eye via adaptive optic elements.

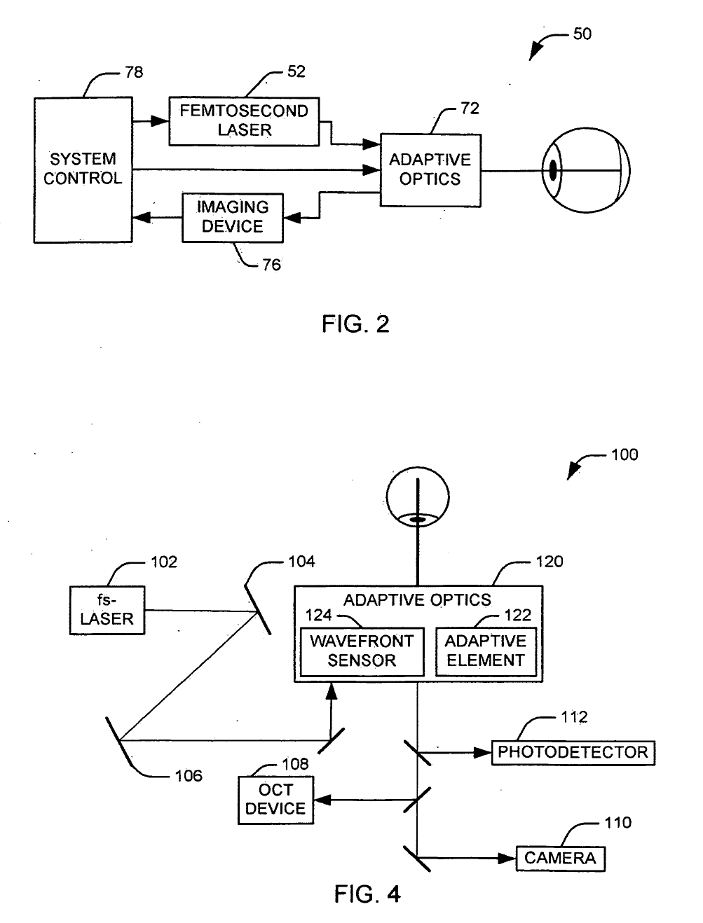

FIG. 2 illustrates a system (50) for precisely applying transcorneal laser pulses to tissue at a target location. The laser pulses are produced by a femtosecond laser (52) that is operative to produce ultra short, high intensity laser pulses. In one implementation, the femtosecond laser is operative to produce pulses having duration between 10 and 1000 femtoseconds and having an intensity in the MW/cm²-GW/cm² range. Table 1 illustrates a range of suitable parameters for the laser in one implementation of the system (20).

TABLE 1

| | minimum | maximum | Typical |
|---|---|---|---|
| pulse duration T | 10 fs | 1000 fs | 50 fs |
| pulse energy E | 100 pJ | 100 nJ | 1 nJ |
| spot diameter d | 0.5 μm | 10 μm | 1 μm |
| intensity I | 130 MW/cm² | 5,000 TW/cm² | 2.5 TW cm² |
| Fluence F | 130 μj/cm² | 50 J/cm² | 130 mJ/cm² |

Pulses of appropriate intensity can induce non-linear absorption in the focus of the laser beam, causing disruption of tissue at the focus while leaving tissue outside of the focus mostly intact. Aberrations within the eye anterior of the target location can cause spatial distortion in the laser beam, resulting in an increase of pulse energy threshold: for photodisruption and corresponding damage to surrounding tissue. Dispersion within the eye anterior of the target location can cause a temporal extension in the laser pulse, providing a second source of distortion resulting in an increase of pulse energy threshold for photodisruption and corresponding damage to surrounding tissue. It has thus been infeasible to direct transcorneal pulses to retinal and preretinal structures in the posterior portion of the eye.

To mitigate aberration of the laser beam within the eye, an adaptive optical element (72) can be utilized. The adaptive optical element (72) can include deformable mirrors, phase modulators, or any other appropriate devices that can be utilized to correct the laser pulse for distortions within the eye, allowing the system (50) to maintain a high optical resolution for the laser pulse at the target location. The adaptive optical element (72) can include a sensor for detecting distortions in a reflected wavefront from the eye. Using feedback from reflected light (e.g., at wavefront sensor or image analysis of all or a portion of an in vivo image of the eye), the reflective properties of the adaptive optical element can be altered to correct for the determined distortion.

During operation, the target location can be imaged by an imaging system (76) to assist an operator in focusing the laser pulses to a desired target location. The imaging system (76) can utilize any appropriate imaging modality for imaging at least the target region of the eye. For example, the imaging element can comprise an optical coherence tomography (OCT) scanner. Additionally or alternatively in one implementation, autofluorescent emissions induced within the eye tissue by the laser pulse can be utilized by the imaging system to produce the desired image data. The data produced by the imaging system (76) can be evaluated at a system control (78) and provided to an operator in a human comprehensible form.

In one implementation, the system control (78) can direct the femtosecond laser (52) to produce multiple pulses, separated by a slight time delay. By selecting pulses having appropriate properties, the interaction between the two pulses can be used to further limit the volume of tissue disrupted by the laser pulses, allowing for increased precision. For example, a first pulse can be produced using a fundamental mode of the laser (52), with a first polarization and an intensity less than a threshold intensity necessary to cause tissue disruption. A second pulse, delayed in time by a short duration (e.g., 20 fs to 1 ps), can be produced using a second, more complex mode of the laser (52), with a polarization perpendicular to the first polarization and an intensity greater than the threshold intensity. The second pulse can be shifted spatially relative to the first pulse, such that a portion of the tissue irradiated by the second pulse is not irradiated by the first pulse. Due to the interaction between the first and second pulses, only tissue that is irradiated by the second pulse but not irradiated by the first pulse will be disrupted. Accordingly, superior resolution can be achieved with the dual pulse arrangement, allowing for increased precision in the ablation of the preretinal or retinal tissue.

FIG. 3 illustrates a graphical representation (80) of the effect of diffraction at the pupil on aberration within the eye. The graph comprises a horizontal axis (82), representing pupil size, and a vertical axis (84), representing visual acuity. The visual acuity of an eye corresponds to and parallels the expected quality of a laser focus. A first line (86) on the graph represents the theoretical diffraction limit of the eye for a given pupil size, and a second line (88) indicates the performance expected for an actual eye. The region (90) bounded by these two lines represents the effect of aberrations in the eye. It will be noted that the effects of these aberrations worsens as the pupil size increases, making any effort at a corrective procedure, such as a viterorectomy, particularly difficult at large pupil sizes. Unfortunately, it is often highly desirable to maximize the pupil size to maximize the numerical aperture (NA) and to minimize the focal laser spot diameter.

FIG. 4 illustrates one example of an implementation of a system (100) for noninvasive ablation of preretinal or retinal tissue within an eye via a transcorneal laser pulse. The system (100) includes a femtosecond laser (102) that is operative to provide high-intensity, low duration pulses to a region in the posterior portion of the eye. In the illustrated example; a laser using an Ti:Sapphire amplifier and an Erbium fiber oscillator can be used to produce pulses that are around hundred femtoseconds in duration and provide between 100 Kilowatts and ten Megawatts of peak power.

A set of scanning mirrors (104) and (106) can be used to aim the laser pulse at a target location within the retina or preretinal region of the eye. It will be appreciated that each of the set of scanning mirrors (104) and (106) is capable of manipulation by a user to shift the focus of the laser pulse in either a horizontal or vertical direction. The targeting of the laser via the scanning mirrors (104) and (106) can be guided by image data provided by one or more of an optical coherence tomography (OCT) scanner (108) and a video camera (110) and a photodetector (112) which detects autofluorescent light exited with the laser pulse by multi photon absorption. For example, the image data can be interpreted and displayed to the user at a user interface (not shown) to facilitate targeting of the laser.

The beam is precorrected for aberrations within the eye at an adaptive optics assembly (120). The adaptive optics assembly comprises an adaptive element (122) that can be manipulated as to adjust its optical properties, such that one or more properties of the laser beam will be altered through interaction with the adaptive element (122). For example, the adaptive element can comprise a deformable mirror or a phase modulator configured for a laser beam of appropriate wavelength and intensity. A wavelength sensor (124) detects distortion in wavefronts reflected from the eye, providing an indication of the aberrations within the eye. The optical properties of the adaptive element (122) can be altered in accordance with the output of the wavefront sensor, such that the laser beam is precorrected for the optical aberrations of the eye, allowing for maintenance of a precise focus despite passage through the cornea and the anterior vitreous matter.

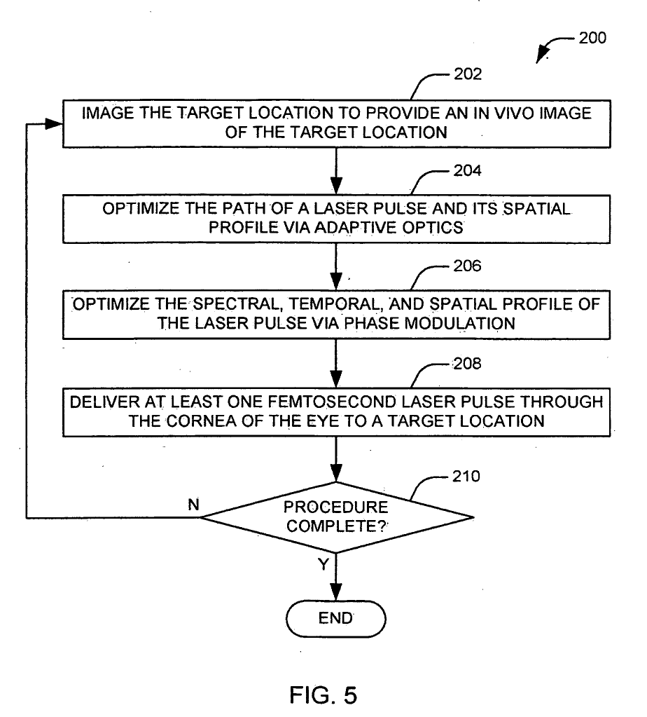

In view of the foregoing structural and functional features described above, methodology in accordance with various aspects of the present invention will be better appreciated with reference to FIG. 5. While, for purposes of simplicity of explanation, the methodology of FIG. 5 is shown and described as executing serially, it is to be understood and appreciated that the present invention is not limited by the illustrated order, as some aspects could, in accordance with the present invention, occur in different orders and/or concurrently with other aspects from that shown and described herein. Moreover, not all illustrated features may be required to implement a methodology in accordance with an aspect the present invention.

FIG. 5 illustrates a method (200) for precisely disrupting tissue within the retina or a preretinal region of the eye in accordance with an aspect of the present invention. At step (202), the target location is imaged via an appropriate imaging modality to produce an in vivo image of the target location. For example, autofluorescent light from the eye tissue induced by the laser pulse can be used to image the location. Alternatively, an optical coherence tomography (OCT) system can be used to produce an image of the area of interest or a conventional video camera can be used to image the area of interest.

At step (204), an adaptive optical element is utilized to optimize the path of the laser pulse within the eye as well as its spectral profile. By selecting an optimal beam path, it is possible to substantially reduce dispersion and aberrations of the laser pulse due irregularities within eye tissue between the surface of the eye and the target location. The adaptive optical element can comprise one or more of a deformable mirror, a phase modulator, and another suitable mechanism for adjusting the optical properties of the laser pulse. At step (206), the spectral, temporal, and spatial profile of the laser pulse is optimized via phase modulation.

At step 208, at least one therapeutic femtosecoond laser pulse is delivered through the cornea of the eye to a target location. In accordance with an aspect of the present invention, the at least one laser pulse has sufficient intensity to induce nonlinear absorption in tissue within the target location. For example, the laser pulses can be high intensity, low duration pulses, having a duration on the order of ten to several hundred femtoseconds.

In one implementation, the at least one laser includes a first laser pulse and a second, time delayed laser pulse at the target location. To make use of the interaction between two laser pulse, the first pulse can be generated, using a fundamental mode of the laser, with a first polarization and an associated intensity less than a threshold intensity necessary for tissue disruption. The second pulse can be generated, using a secondary, more complex mode of the laser, with a second polarization that is perpendicular to the first polarization and an associated intensity that is greater than the threshold intensity. The pulses can be separated spatially, such that a portion of the tissue at the target location will be, irradiated by the second laser pulse, but not by the first laser pulse. In this manner, the interaction between the pulses can be utilized to further narrow the focus of the laser, as only the portion of the tissue at the target location that is irradiated by the second laser pulse but not by the first laser pulse will be ablated.

At step 210, it is determined if the procedure is complete. If not (N), the methodology 200 returns to 202 to generated a new image of the target area. If the procedure is complete, (Y) the methodology terminates.

It will be understood that the above description of the present invention is susceptible to various modifications, changes and adaptations, and the same are intended to be comprehended within the meaning and range of equivalents of the appended claims. The presently disclosed embodiments are considered in all respects to be illustrative, and not restrictive. The scope of the invention is indicated by the appended claims, rather than the foregoing description, and all changes that come within the meaning and range of equivalence thereof are intended to be embraced therein.

Having described the invention, the following is claimed:

1. A method for disrupting tissue within preretinal and retinal structures of an eye, comprising:
directing at least one femtosecond laser pulse through the cornea of an eye to a target location, the at least one femtosecond laser pulse having sufficient intensity to induce nonlinear absorption in tissue within the target location;
correcting the at least one laser pulse using an adaptive optical element as to substantially reduce aberration and dispersion of the at least one laser pulse due to changes in an ocular wavefront profile of the laser pulse during transmission through eye tissue between the surface of the eye and the target location;
imaging at least the target location to produce an in vivo image; and
adjusting the adaptive optical element according to distortion detected in a reflected wavefront.

2. The method of claim 1, wherein adjusting the adaptive optical element comprises activating at least one actuator associated with one of a deformable mirror and a phase modulator according to the detected distortion.

3. The method of claim 1, wherein imaging at least the target location comprises obtaining an optical coherence tomography image of at least the target location.

4. The method of claim 1, wherein the at least one laser pulse has a duration of between 10 and 1000 femtoseconds.

5. The method of claim 1, wherein directing at least one laser pulse at the target location, comprises directing first laser pulse and a second, time delayed laser pulse at the target location.

6. The method of claim 5, the first pulse having a first polarization and the second pulse having a second polarization that is perpendicular to the first polarization.

7. The method of claim 5, wherein the first pulse has an associated intensity less than a threshold intensity necessary for tissue disruption and the second pulse has an associated intensity greater than the threshold intensity.

8. The method of claim 5, wherein the first pulse is produced via a first mode associated with a laser producing the first and second pulses, and the second pulse is produced via a second mode of the laser.

9. The method of claim 8, wherein the first mode is a fundamental mode of the laser and the second mode is a secondary mode.

10. The method of claim 8, wherein the first pulse has a first polarization and an associated intensity less than a threshold intensity necessary for tissue disruption, and the second pulse has a second polarization that is perpendicular to the first polarization and an associated intensity that is greater than the threshold intensity, such that only a portion of the tissue at the target location that is irradiated by the second laser pulse but not by the first laser pulse will be ablated.

11. The method of claim 1, wherein imaging at least the target location comprises obtaining an autofluorescent image of at least the target location.

12. A system for precisely disrupting tissue within preretinal and retinal structures of the eye, comprising:
a femtosecond laser configured to direct laser pulses having a duration on the order of femtoseconds through the cornea of the eye to a target location in the preretinal vitreous tissue or retinal microstructure;
an imaging element operative to image at least the target location to produce an in vivo image; and
an adaptive optical element operative to correct laser pulses from the laser apparatus as to substantially compensate for the effects of optical aberrations and dispersion within eye tissue anterior of the target location.

13. The system of claim 12, the femtosecond laser being configured to produce a laser pulse providing between 0.1 to 100 Nanojoules of energy.

14. The system of claim 12, the femtosecond laser being configured to produce a laser pulse having a wavelength between 400 nm and 1400 nm.

15. The system of claim 12, the adaptive optical element comprising:
an adaptive element that can be manipulated as to adjust its optical properties, such that one or more properties of the laser pulse will be altered through interaction with the adaptive element; and
a wavefront sensor that detects distortion in wavefronts reflected from the eye to provide an indication of optical aberrations within the eye, such that the optical properties of the adaptive element be altered in accordance with the output of the wavefront sensor.

16. The system of claim 12, further comprising a system control that directs the femtosecond laser to direct a first laser pulse and a second, time delayed laser pulse at the target location, the system control configuring the laser as to produce an interaction between the first pulse and the second pulse, such that only a portion of the tissue at the target location that is irradiated by the second laser pulse but not by the first laser pulse will be ablated.

17. An apparatus for precisely disrupting tissue within preretinal and retinal structures of the eye, comprising:
a femtosecond laser configured to direct laser pulses having a duration on the order of femtoseconds through the cornea of the eye to a target location in the preretinal vitreous tissue or retinal microstructure; and
an adaptive optical element operative to correct laser pulses from the laser apparatus as to substantially mitigate the effects of optical aberrations within eye tissue anterior of the target location, the adaptive optical element comprising:
an adaptive element that can be manipulated as to adjust its optical properties, such that one or more properties of the laser pulse will be altered through interaction with the adaptive element; and
a wavefront sensor that detects distortion in wavefronts received from the eye to provide an indication of optical aberrations within the eye, such that the optical properties of the adaptive element be altered in accordance with the output of the wavefront sensor.

18. The apparatus of claim 17, further comprising an imaging element operative to image at least the target location from autofluorescent light induced by laser pulses.

19. The apparatus of claim 17, the adaptive element comprising a deformable mirror.

20. The apparatus of claim 17, the adaptive element comprising a phase modulator.
